(19) United States
(12) Patent Application Publication
(10) Pub. No.: US 2014/0094699 A1
(43) Pub. Date: Apr. 3, 2014

(54) PROCESS FOR PRODUCING A RADIOLOGY REPORT

(71) Applicant: Jefferson Radiology, P.C., (US)

(72) Inventors: Robert M. SPILLANE, West Hartford, CT (US); Aimee McDANIEL-LAUT, South Glastonbury, CT (US); Ethan B. FOXMAN, West Hartford, CT (US); Theresa E. SZYMANSKI, West Hartford, CT (US)

(73) Assignee: JEFFERSON RADIOLOGY, P.C., East Hartford, CT (US)

(21) Appl. No.: 13/799,352

(22) Filed: Mar. 13, 2013

Related U.S. Application Data
(60) Provisional application No. 61/687,096, filed on Apr. 18, 2012.

(51) Int. Cl. A61B 8/08

(52) U.S. Cl. CPC A61B 8/5292
USPC 600/437

(57) ABSTRACT

A process for manufacturing a radiology report wherein a first technologist prepares a worksheet that records objective data from the image display of the condition of a patient's body that was subjected to a controlled source of imaging energy. A second different technologist prepares a preliminary report in narrative form from the work sheet prepared by the first technologist and image display. A radiologist uses the preliminary report and the image to prepare a final report.

### CROSS-REFERENCE TO RELATED APPLICATION

This application claims the benefit of U.S. Provisional Patent Application No. 61/687,096 entitled “Process For Producing a Radiology Report”, filed Apr. 12, 2012, the disclosure of which is incorporated herein by reference in its entirety.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### TECHNICAL FIELD

This application invention relates to the general field of medical diagnostics and intervention and, in particular, to the preparation of radiology reports based on the imaging of portions of a patient's body.

### BACKGROUND

One important medical specialty is the field of radiology whereby various forms of energy are available to interact with body organs or tissue and produce an image that can be analyzed and interpreted by a radiologist. Generally, a primary care physician who suspects that a patient may have a particular ailment that could be diagnosed through radiology, will refer the patient to a radiology facility which is equipped to handle a relatively large volume of patients. The typical process involves the steps of determining the type of imaging that has been requested by the primary physician, preparing a protocol that the imaging technologist uses to select and position the imaging equipment, exposing a portion of the patient's body to the controlled source of imaging energy and, for some types of imaging, preparing a worksheet containing objective data observed from images derived from the exposures or scans. The worksheet is forwarded to a radiologist who inspects at least the worksheet and images for which a radiology report will be prepared. Although the report could be prepared as a free-form narrative, it is common practice that the radiologist has a voice recognition program including a template that is appropriate for the particular type of image such that the radiologist can be prompted to follow a standard sequence of findings concerning the image. The template includes prompts for the radiologist to provide a detailed interpretation of the image, and thereby complete the report.

The most important part of the report is the image interpretation. Image interpretation requires the highest degree of training, skill, and experience, but the radiologist wastes valuable time in preparing the entire report.

### SUMMARY

An object of the method disclosed herein is to improve the production process, including efficiency and quality, of radiology reports, by providing an intermediary technologist who can prepare a preliminary report from the imaging worksheet. The radiologist can review and supplement the preliminary report with the image interpretation to produce a final report.

From one aspect, the method is directed to a process in which a first technologist exposes a portion of the patient's body to a controlled source of imaging energy, and from the interaction of the imaging energy with the body, equipment generates an image display that represents a condition of that portion of the patient's body. From the image display, the first technologist prepares a work sheet that records objective data about the condition of that portion of the body. From the work sheet and the image display, a second technologist transforms the objective data on the work sheet into a series of narrative findings in a preliminary report. From the image display and the preliminary report, a radiologist analyses the displayed image and converts the preliminary report into a final report by adding findings including a diagnostic interpretation.

From a generalized perspective, one can consider that the first, imaging technologist, records on the work sheet specified gross properties including quantitative data such as size measurements as well as normal and seemingly abnormal condition. The second, quality, technologist evaluates the gross properties and records associated sub-properties as findings and also confirms/and or flags other potentially abnormal conditions. The radiologist draws and records medically diagnostic interpretations and conclusions from the work product of the imaging technologist and the quality technologist.

Preferably, the second technologist prepares the preliminary report using a voice recognition computer program which generates a template that displays a series of field headings and empty data fields into which the second technologist dictates a combination of quantitative and narrative entries, and the radiologist uses the same voice recognition computer program to dictate the diagnostic interpretation into a dedicated field in the preliminary report.

To our knowledge, no previous process or procedure included the intermediate step of a second technologist with intermediate skills transforming objective data on a worksheet from the imaging technologist into findings in a preliminary radiology report that need only be supplemented with the high value interpretation of the radiologist.

### BRIEF DESCRIPTION OF THE DRAWING

One non-limiting example of the invention will be described with reference to the accompanying drawing, in which:

FIG. 1 is a schematic representation of the flow of documents and information to and from the quality technologist;

FIG. 2 is a representative requisition form which specifies the purpose of the imaging and some demographic information about the patient;

FIG. 3 is a sample worksheet to be filled in by the imaging technologist as a result of operating the imaging equipment;

FIG. 4 is a sample of a preliminary report prepared by the quality technologist; and

FIG. 5 is a sample of the final report prepared by the radiologist based on the preliminary report of FIG. 4.

### DETAILED DESCRIPTION

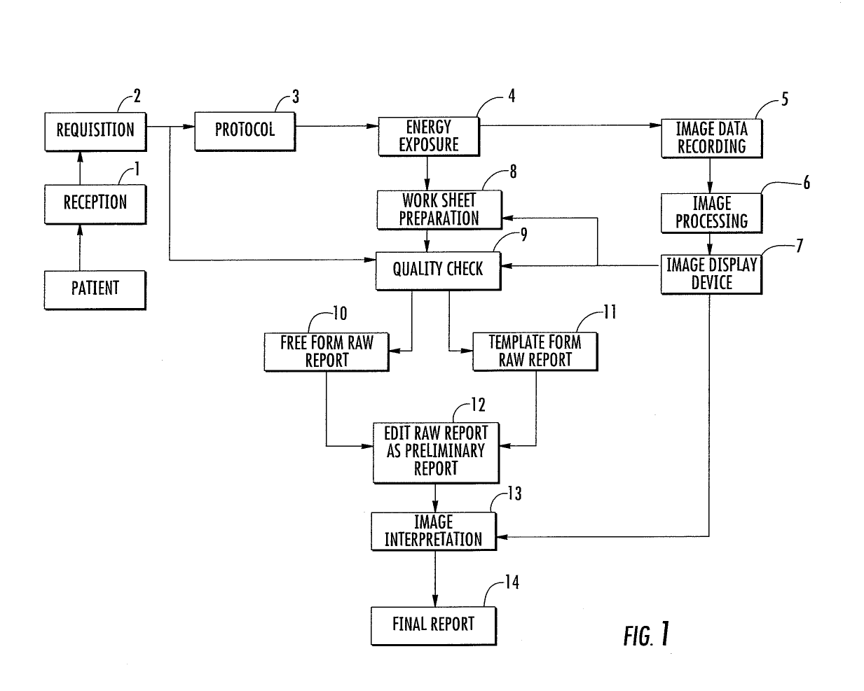

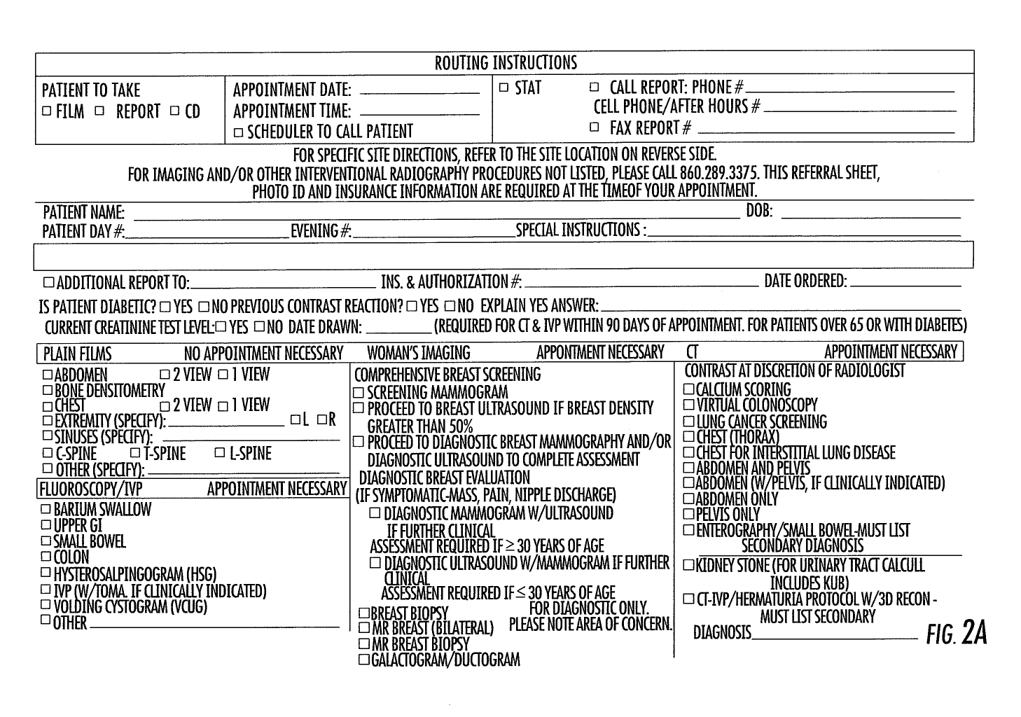

FIG. 1 depicts the process for manufacturing a radiology image report on a particular patient for a particular purpose at a radiological office or facility.

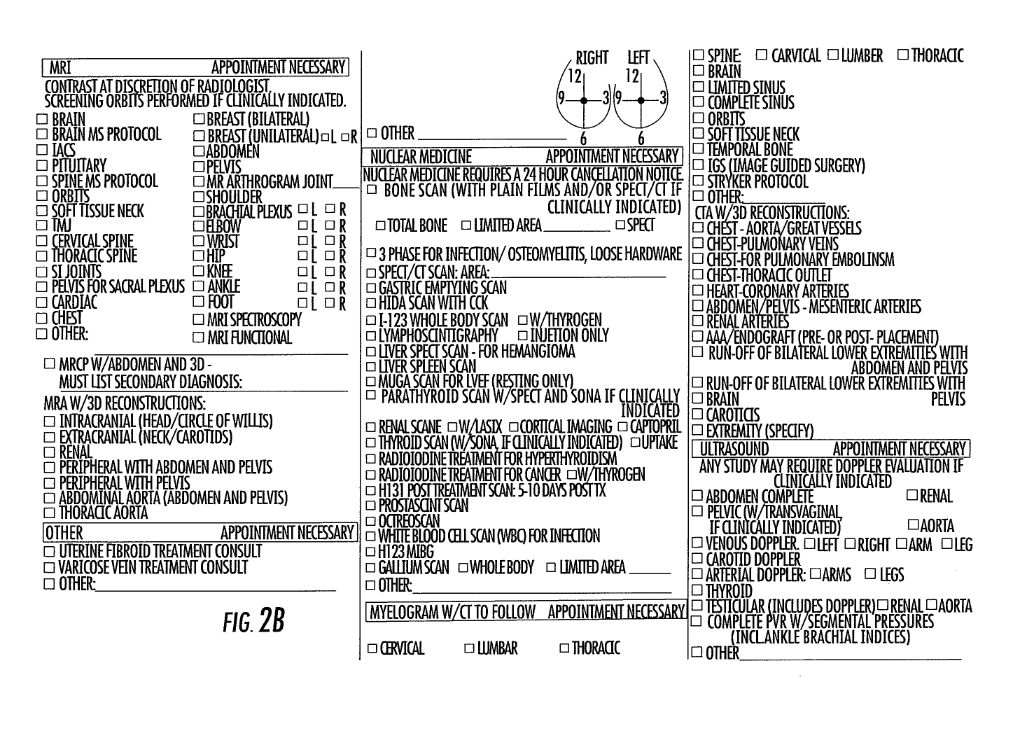

As indicated in blocks 1 and 2, a receptionist or similar patient intake processor enters data into the system from a requisition document brought into the office by a patient, sent to the office by the primary physician, or prepared while a patient is in consultation with a doctor at the facility. Information on a requisition can take a variety of forms, with one example shown in FIG. 2. The upper portion contains demographic data that is associated with the patient, the middle portion is a summary of the symptoms or the like and is signed by a physician, and the lower portion has multiple boxes corresponding to the categories of imaging techniques based on the type, detection and processing use of the energy to be used. Within each of those categories is a check list of the body or particular organs or tissue that is to be subjected to the scan.

According to any of a variety of procedures available in this field, one or more persons at the facility prepare or decide, as appropriate, upon a protocol as indicated at block 3, e.g., such as a complete ultrasound versus a limited ultrasound scan. The imaging technologist then positions the patient for energy exposure as indicated in block 4, according to the requisition and protocol. The equipment records image data as indicated at 5, includes algorithms for processing the data to extract an image data file as indicated in block 6, and produces a display on a monitor or film as indicated at block 7. The display is in the form of a diagnostic image of contrasting color or gray scale values that represent a condition of the organs and/or tissue in the scanned portion of the patient's body.

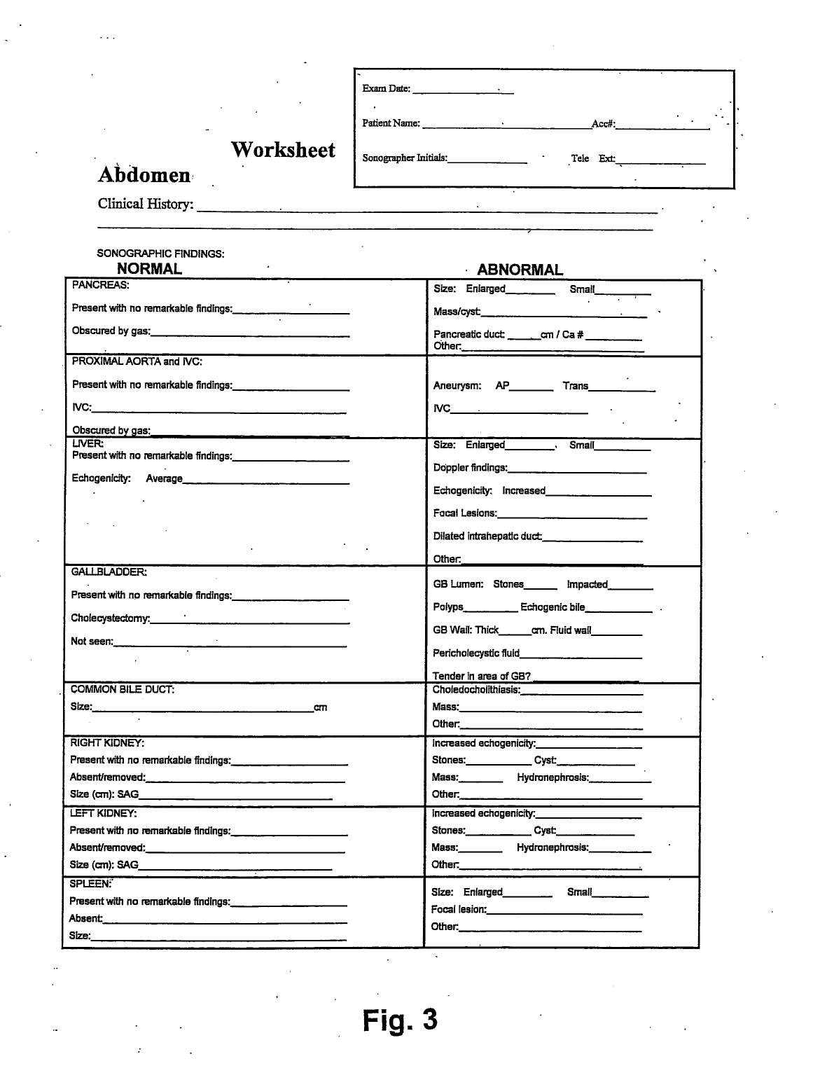

In the particular example of a sonogram of a complete abdomen, the imaging technologist would prepare a worksheet at block 8 (FIG. 1) such as shown in FIG. 3, from the protocol 3 and the displayed image 7, preferably with a cross check against the requisition 2.

In addition to patient demographic information, accession number, and other information from the requisition, the worksheet as shown in FIG. 3 has a left column for the imaging technologist to enter size or other objective data and whether the organ or tissue or condition appears normal. The entries under the normal column can include present with no remarkable findings; absent or removed; size; or obscured. In the right side, the imaging technologist enters data and other objective information for conditions that appear abnormal. These can include qualitative or quantitative indications of size, dilation, excess fluid, or other information which would prompt the radiologist to analyze the image more closely in the course of interpreting the images.

According to the present invention, the worksheet prepared at block 8 and image from block 7 are not forwarded to the radiologist at this time, but rather to a second technologist associated with actions taken in block 9, who has the training and experience to prepare a preliminary version of the report that the radiologist will ultimately furnish to the primary physician or other physician who requested the radiological diagnosis.

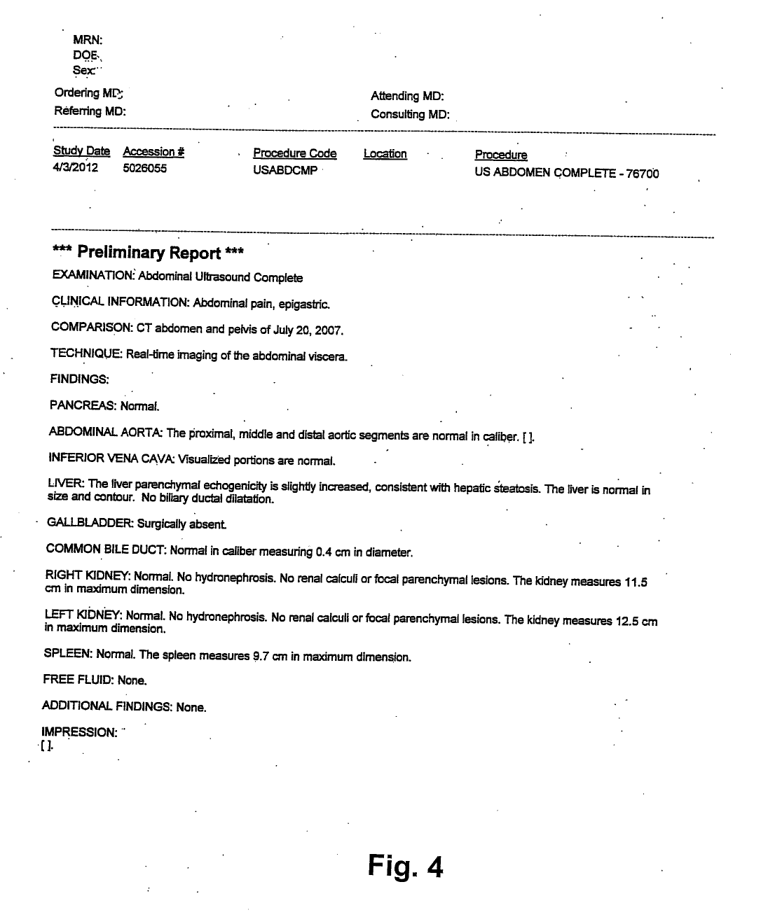

FIG. 4 is a sample of a preliminary report prepared by a quality technologist based on a worksheet such as shown in FIG. 3 for a complete ultrasound abdominal scan. Although this report could be completely free-form as indicated in block 10, in the sense of a running narrative, either typed, dictated for transcription, or entered into a voice recognition system (each of which is in conventional use by radiologists), the preferred option as indicated in block 11, is for the quality technologist to call up a voice recognition system that has a pre-established template associated with each protocol. In the example shown in FIG. 4, the prompts are shown in capital letters, each prompt appearing as the voice entry is completed for the preceding prompt. The template prompts for patient and administrative information derived from the requisition and internal procedure and accession numbers. Substantively, the prompts include a series of field headings and associated empty data fields corresponding to various findings. Typically, the template system includes the option of selecting from an array of stock sentences or phrases that can be entered via voice selection or via a mouse or similar device.

It is evident from FIG. 1 that the quality check indicated in block 9 by the quality technologist is based on input from the requisition block 2, the image display from block 7, and the worksheet from block 8 (which should be accompanied by protocol information or this can be accessed directly from block 3).

Importantly, the IMPRESSION field remains blank in the preliminary report, as this is the field where the radiologist will enter the diagnostic information, which is the critical component of the final report.

Inspection of the preliminary report in FIG. 4 shows that for the ABDOMINAL AORTA FIELD the complete sentence indicates a normal condition. However, a pair of brackets “[ ]” also appears. The quality technologist enters these brackets in any field where the worksheet (FIG. 3) has an entry in the abnormal column. Stated differently, any entry in the abnormal column of a worksheet prompts the quality technologist to enter a pair of brackets in the corresponding findings field in the preliminary report. In some instances, such as the field for the abdominal aorta, a standard phrase is indicated for one kind of normality, and a pair of brackets is also entered to indicate the possible presence of some kind of anomaly in the image. in other instances, a given field may have only a pair of brackets.

Especially when prepared with voice recognition, the raw report often needs editing and this is also performed by the quality technologist as indicated in block 12, thereby saving the radiologist's valuable time.

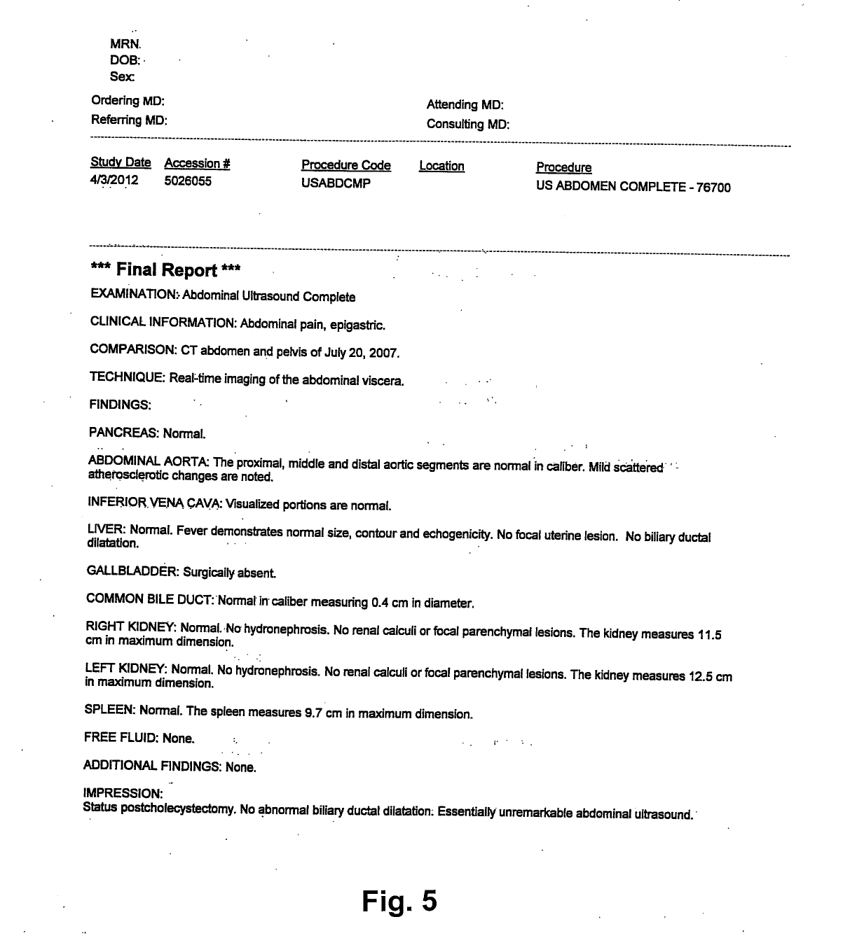

Upon editing the raw report as indicated in block 12, the preliminary report is made available to the radiologist for image interpretation as indicated in block 13. As is evident from FIG. 5, the radiologist has supplemented the preliminary report in two respects. First, with respect to the pair of brackets shown in the abdominal aorta field in FIG. 4, the radiologist has entered the finding, “Mild scattered atherosclerotic changes are noted”. In the impression field, the radiologist has entered, “Status postcholecystectomy. No abnormal biliary ductal dilation. Essentially unremarkable abdominal ultrasound.” The preliminary report may also include fields for entry of the time at which the preliminary report was prepared, the person who prepared the preliminary report, the time at which the final report was prepared, and the radiologist who interpreted the image and added the impressions and signed the final report. In this process, the preliminary report can be saved in digital form until accessed and in essence revised by the radiologist and then saved only as a revised, final document. Alternatively, both the preliminary report and the revised (final) report can be saved as distinct documents.

Preferably, the requisition, preliminary report and image are available to the radiologist at the same place and at the same time as a last step for identifying any anomalies or inconsistencies in any field. However, the central concept of the present invention is that a qualified technologist is interposed between the imaging technologist and radiologist to remove the need for the radiologist to check all fields and thereby to significantly lessen the time spent by the radiologist in reviewing the compiled information. Both the imaging technologist and the quality technologist would typically have at least two years of specialized education or training, and would be appropriately certified. In general, each imaging technologist would have specialized capability in one or two of the categories of energy imaging such as fluoroscopy, MRI, nuclear medicine, ultrasound, or the like. The quality technologist could likewise have special capability in one or two of these areas, preferably having practiced previously or concurrently as an imaging technologist. On the other hand, the quality technologist could acquire appropriate training to perform the functions described herein for many if not all of the categories of radiology scans shown in the requisition form (FIG. 2).

Although the present invention relates to the field of medicine, the process of interest can be viewed as a process for manufacturing a radiology imaging report. The process is preferably implemented with a computer network that is accessible by the receptionist per block 1, the imaging technologist per block 8, the quality technologist per blocks 9, 10, 11, and 12, and the radiologist per blocks 13 and 14. The preparation of the final report requires multiple stages of transformation of data. For example, a paper requisition such as shown in FIG. 2 is converted into digital form on the server at block 2, for access and display on a monitor available to several others in downstream operations. The information on the internal worksheet prepared per block 8 is transformed into a report having a format and findings suitable for sending to the referring physician, by an entity at blocks 9-12 that is completely new in this field, i.e., a person who preferably has an intermediate level of training between an imaging technologist and a medical doctor.

The responsibility of the quality technologist requires exercising a degree of skill and judgment that falls between those of the imaging technologist and the radiologist. The imaging technologist has the training to set up and operate the equipment in accordance with the protocol, inspect the images, and record on the work sheets (such as FIG. 3) objective properties or conditions as observed on the images. The quality technologist has the somewhat more sophisticated responsibility of transforming the objective data on the work sheet into a more qualitative summary or narrative as contained in the preliminary report (FIG. 4).

Generally the imaging technologist would check that the diagnostic image corresponds to the particular patient, the diagnostic image is consistent with the protocol and shows the condition of the portion of the body corresponding to the purpose of the imaging, and the diagnostic image has a sufficient range of contrast and discernible contrast borders to permit interpretation by a radiologist. Preferably, the quality technologist also performs a similar check, e.g., that that the diagnostic image corresponds to the particular patient, the diagnostic image is consistent with the protocol and worksheet and shows the condition of the portion of the body corresponding to the purpose of the imaging, and the diagnostic image has a sufficient range of contrast and discernible contrast borders to permit interpretation by a radiologist. The quality technologist can also check for missing information on the images, missing or incorrect measurements, missing information that wasn't recorded on the worksheets as well as the accuracy of the information recorded on the worksheets.

Whereas the recording choices available for the imaging technologist are narrowly constrained by the relatively few prompts on the work sheet, the sentences and phrases available to the quality technologist are much more varied and detailed. For example, in a template based report generation system having pre-established “findings” fields to be filled in by typing or voice recognition, the template associated with each protocol provides many options for each field, and the quality technologist is free to enter original observations or comments. Internal procedure may require that the quality technologist include a flag or the like (such as the brackets [ ]) in conjunction with any original observations. One commercial voice recognition system that is adaptable (e.g., by adding the “[ ]” or similar indicia) for use by both the quality technologist and the radiologist according to the invention, is available under the trademark Powerscribe from Nuance Communications, Inc. of Burlington, Vt.

It should be appreciated that in the preferred implementation of the system associated with FIG. 1, the work sheet (FIG. 3), preliminary report (FIG. 4), processed image, and requisition data are all in digital form with a common searchable attribute such as accession number or the like, whereby the radiologist can assemble any or all of these documents for viewing at one display device to prepare the final report (FIG. 5). In the least automated implementation, all documents are on physical media, such as paper or film, and these documents are passed along sequentially from the imaging technologist to the quality technologist to the radiologist.

While a preferred embodiment has been shown and described, various modification and substitutions may be made thereto. Accordingly, it is understood that the present embodiment has been described by way of illustration and not limitation.

1. A process for manufacturing a radiology image report on a particular patient, comprising:
exposing a portion of the patient's body to a controlled source of imaging energy;
from the interaction of the imaging energy with the body, generating an image display that represents a condition of that portion of the patient's body;
from the image display, preparing a work sheet by a first technologist that records objective data about the condition of that portion of the body;
from the work sheet and the image display, transforming the objective data on the work sheet into a series of narrative findings in a preliminary report by a second technologist; and
analyzing the displayed image and converting the preliminary report into a final report by adding findings including a diagnostic interpretation by a radiologist.

2. The process of claim 1, wherein
the first technologist records quantitative data and gross properties including normal and seemingly abnormal condition;
the second technologist prepares the preliminary report by filling in a plurality of predefined fields with narrative phrases concerning the normal conditions from the work sheet and in at least one field entering an indication of an abnormal condition from the work sheet.

3. The process of claim 2, wherein the second technologist prepares the preliminary report using a voice recognition computer program which generates a template that displays a series of field headings and empty data fields into which the second technologist dictates a combination of quantitative and narrative entries; and
the radiologist uses the same voice recognition computer program and dictates the diagnostic interpretation into a dedicated field in the preliminary report.

4. A process for manufacturing a radiology image report on a particular patient, comprising the steps whereby:
a first technologist exposes a portion of the patient's body to a controlled source of imaging energy;
from the interaction of the imaging energy with the body, equipment generates an image display that represents a condition of that portion of the patient's body;
from the image display, the first technologist prepares a work sheet that records objective data about the condition of that portion of the body;
from the work sheet and the image display, a second technologist transforms the objective data on the work sheet into a qualitative summary in a preliminary report;
from the image display and the preliminary report, a radiologist analyzes the displayed image and converts the preliminary report into a final report by adding a diagnostic interpretation.

5. The process of claim 4, wherein
the first technologist records quantitative data and gross properties including normal and seemingly abnormal condition;
the second technologist prepares the preliminary report by filling in a plurality of predefined fields with narrative phrases concerning the normal conditions from the work sheet and in at least one field entering an indication of an abnormal condition from the work sheet.

6. The process of claim 5, wherein
the second technologist prepares the preliminary report using a voice recognition computer program which generates a template that displays a series of field headings and empty data fields into which the second technologist dictates a combination of quantitative and narrative entries; and
the radiologist uses the same voice recognition computer program and dictates the diagnostic interpretation into a dedicated field in the preliminary report.

7. A process for manufacturing a radiology image report on a particular patient, comprising the steps whereby:
an imaging technologist exposes a portion of the patient's body to a controlled source of imaging energy;
image data are recorded commensurate with the interaction of the imaging energy with an organ or tissue in that portion of the patient's body;
the image data are processed to display a diagnostic image of contrasting color or gray scale values that represents a condition of the organ or tissue in that portion of the patient's body;
the imaging technologist prepares a work sheet that records quantitative data and gross properties including normal and seemingly abnormal condition about the organ or tissue;
from the work sheet and the displayed diagnostic image, another technologist prepares a preliminary report of findings; and
the displayed diagnostic image and the preliminary report are presented to a radiologist, who interprets the displayed diagnostic image and converts the preliminary report into a final report by adding a diagnostic interpretation.

8. A process for manufacturing a radiology image report on a particular patient for a particular purpose, comprising:
recording patient requisition data including demographic data and imaging purpose data to produce a procedure protocol;
according to the protocol, exposing a portion of the patient's body to a controlled source of imaging energy;
recording image data commensurate with the interaction of the imaging energy with an organ or tissue in that portion of the patient's body;
processing the image data to display a diagnostic image of contrasting color or gray scale values that represents a condition of the tissue in that portion of the patient's body;
said first technologist preparing a work sheet that records quantitative data and gross properties including normal and seemingly abnormal condition about the organ or tissue;
from inspection of the requisition data, the work sheet, and the displayed diagnostic image, a different, second technologist
(i) performing a quality check including,
that the diagnostic image corresponds to the particular patient,
that the diagnostic image is consistent with the work sheet and shows the condition of the portion of the body corresponding to the purpose of the imaging, and
that the diagnostic image has a sufficient range of contrast and discernible contrast borders to permit interpretation by a radiologist; and
(ii) transforming the quantitative data and gross properties including normal and seemingly abnormal condition on the work sheet into a qualitative summary in a preliminary report; and
presenting the requisition data, the work sheet, the diagnostic image, and the preliminary report to a radiologist, who converts the preliminary report into a final report by adding an interpretation to the preliminary report.
